(19) United States
(12) Patent Application Publication
(10) Pub. No.: US 2021/0269530 A1
(43) Pub. Date: Sep. 2, 2021

(54) CONDITIONALLY ACTIVATED BINDING PROTEIN COMPRISING A STERICALLY OCCLUDED TARGET BINDING DOMAIN

(71) Applicant: HARPOON THERAPEUTICS, INC., South San Francisco (CN)

(72) Inventors: Shuoyen Jack LIN, San Bruno, CA (US); Richard J. AUSTIN, San Francisco, CA (US); Bryan D. LEMON, Mountain View, CA (US); Holger WESCHE, San Francisco, CA (US)

(73) Assignee: Harpoon Therapeutics, Inc., South San Francisco, CA (US)

(21) Appl. No.: 17/055,103

(22) PCT Filed: May 14, 2019
(86) PCT No.: PCT/US2019/032306

§ 371 Date: Nov. 12, 2020

Related U.S. Application Data
(60) Provisional application No. 62/756,498, filed on Nov. 6, 2018.
(60) Provisional application No. 62/671,355, filed on May 14, 2018.

(51) Int. Cl. C07K 16/28; C07K 16/30

(52) U.S. Cl. CPC C07K 16/2863; A61K 2039/505; C07K 16/30

(57) ABSTRACT

Disclosed herein is a conditionally active target binding protein that contains a first binding domain that binds to a bulk serum protein and sterically occludes binding of a second binding domain to its target. Pharmaceutical compositions comprising the conditionally active binding proteins disclosed herein and methods of using such compositions are further provided.

### CROSS-REFERENCE

This application claims the benefit of U.S. Provisional Application Nos. 62/671,355 filed May 14, 2018 and 62/756,498 filed Nov. 6, 2018, each of which is incorporated by reference herein in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

There is a need to extend the half-life of a therapeutic, diagnostic, or imaging molecule in circulation and also improve its ability to reach its target within an intended location (e.g., a tumor cell) without non-specific binding.

### SUMMARY OF THE INVENTION

One embodiment provides a conditionally activated binding protein, comprising, in an inactive form: (i) a first binding domain that is capable of binding a bulk serum protein; (ii) a second binding domain that is sterically occluded from binding a target; and (iii) a cleavable linker connecting the first and the second binding domains, wherein upon cleavage of the cleavable linker the binding protein is activated and the second binding domain is capable of binding the target. In some embodiments, the bulk serum protein comprises albumin, transferrin, IgG1, IgG2, IgG4, IgG3, IgA monomer, Factor XIII, Fibrinogen, IgE, pentameric IgM, any variants thereof, any fragments thereof, or a fusion protein comprising any combination thereof. In some embodiments, the first binding domain is bound to the bulk serum protein. In some embodiments, the inactive form the bulk serum protein is in close proximity to the second binding domain, thereby sterically occluding the second binding domain from binding its target. In some embodiments, the first and the second binding domains are connected by a protease cleavable linker. In some embodiments, the cleavable linker comprises a protease cleavage site. In some embodiments, the first binding domain comprises two or more polypeptides linked by a non-cleavable linker. In some embodiments, the binding protein is converted to the activated form upon a cleavage of the cleavable linker, and wherein in the activated form the second binding domain is separated from the first binding domain bound to the bulk serum protein, thereby removing the steric occlusion. In some embodiments, the binding protein is converted to the activated form in a protease rich environment. In some embodiments, the first binding domain comprises a natural peptide, a synthetic peptide, an engineered scaffold, an engineered bulk serum protein, an immunoglobulin, any variants thereof, any fragments thereof, or a fusion protein comprising any combination thereof. In some embodiments, the engineered scaffold comprises at least one of: an sdAb, an scFv, an Fab, a VHH, a IgNAR, a VH, a VL, a fibronectin type III domain, an immunoglobulin-like scaffold, a bacterial albumin-binding domain, an adnectin, a monobody, an affibody, an affilin, an affimer, an affitin, an alphabody, an anticalin, an avimer, a centyrin, a DARPin, a cystine knot peptide, a lipocalin, a three-helix bundle scaffold, a protein G-related albumin-binding module, a DNA or RNA aptamer scaffold, or any combinations thereof. In some embodiments, the first binding domain comprises a binding site specific for the bulk serum protein. In some embodiments, the first binding domain comprises a binding site specific for an immunoglobulin light chain. In some embodiments, the immunoglobulin light chain is an Igκ free light chain. In some embodiments, the first binding domain comprises one or more complementary determining regions (CDRs), and wherein the CDRs provide the binding site specific for the bulk serum protein or the immunoglobulin light chain. In some embodiments, the first binding domain comprises a sequence selected from SEQ ID Nos.: 44-52. In some embodiments, the second binding domain comprises an immunoglobulin molecule or a non-immunoglobulin molecule. In some embodiments, the second binding domain comprises an immunoglobulin molecule, wherein the immunoglobulin molecule is an antibody or an antibody fragment. In some embodiments, the second binding domain comprises a monoclonal antibody, a bispecific antibody, a chimeric antibody, a human antibody, a humanized antibody, a camelized antibody, or a variant thereof. In some embodiments, the second binding domain comprises the antibody fragment, and wherein the antibody fragment comprises a sdAb, Fab, Fab′-SH, Fv, scFv, (Fab′)2 fragment, a fragment of a chimeric antibody, a fragment of a bispecific antibody, or a variant thereof. In some embodiments, in the inactive form the bulk serum protein is in close proximity to a binding site within the second binding domain, wherein the binding site is specific for the target. In some embodiments, the target comprises a tumor antigen. In some embodiments, the tumor antigen comprises at least one of: EpCAM, EGFR, HER-2, HER-3, c-Met, FoIR, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, or WT1. In some embodiments, the tumor antigen comprises at least one of: EpCAM (exemplary protein sequences comprises UniProtkB ID No. P16422, B5MCA4), EGFR (exemplary protein sequence comprises UniProtkB ID No. P00533), HER-2(exemplary protein sequence comprises UniProtkB ID No. P04626), HER-3(exemplary protein sequence comprises UniProtkB ID No. P21860), c-Met (exemplary protein sequence comprises UniProtkB ID No. P08581), FoIR (exemplary protein sequence comprises UniProtkB ID No. P15238), PSMA (exemplary protein sequence comprises UniProtkB ID No. Q04609), CD38 (exemplary protein sequence comprises UniProtkB ID No. P28907), BCMA (exemplary protein sequence comprises UniProtkB ID No. Q02223), and CEA (exemplary protein sequence comprises UniProtkB ID No. P06731, 5T4 (exemplary protein sequence comprises UniProtkB ID No. Q13641), AFP (exemplary protein sequence comprises comprises UniProtkB ID No. P02771), B7-H3 (exemplary protein sequence comprises UniProtkB ID No. Q5ZPR3), CDH-6 (exemplary protein sequence comprises UniProtkB ID No. P97326), CAIX (exemplary protein sequence comprises UniProtkB ID No. Q16790), CD117 (exemplary protein sequence comprises UniProtkB ID No. P10721), CD123 (exemplary protein sequence comprises UniProtkB ID No. P26951), CD138 (exemplary protein sequence comprises UniProtkB ID No. P18827), CD166 (exemplary protein sequence comprises UniProtkB ID No. Q13740), CD19 (exemplary protein sequence comprises UniProtkB ID No. P15931), CD20 (exemplary protein sequence comprises UniProtkB ID No. P11836), CD205 (exemplary protein sequence comprises UniProtkB ID No. 060449), CD22 (exemplary protein sequence comprises UniProtkB ID No. P20273), CD30 (exemplary protein sequence comprises UniProtkB ID No. P28908), CD33 (exemplary protein sequence comprises UniProtkB ID No. P20138), CD352 (exemplary protein sequence comprises UniProtkB ID No. Q96DU3), CD37 (exemplary protein sequence comprises UniProtkB ID No. P11049), CD44 (exemplary protein sequence comprises UniProtkB ID No. P16070), CD52 (exemplary protein sequence comprises UniProtkB ID No. P31358), CD56 (exemplary protein sequence comprises UniProtkB ID No. P13591), CD70 (exemplary protein sequence comprises UniProtkB ID No. P32970), CD71 (exemplary protein sequence comprises UniProtkB ID No. P02786), CD74 (exemplary protein sequence comprises UniProtkB ID No. P04233), CD79b (exemplary protein sequence comprises UniProtkB ID No. P40259), DLL3 (exemplary protein sequence comprises UniProtkB ID No. Q9NYJ7), EphA2 (exemplary protein sequence comprises UniProtkB ID No. P29317), FAP (exemplary protein sequence comprises UniProtkB ID No. Q12884), FGFR2 (exemplary protein sequence comprises UniProtkB ID No. P21802), FGFR3 (exemplary protein sequence comprises UniProtkB ID No. P22607), GPC3 (exemplary protein sequence comprises UniProtkB ID No. P51654), gpA33 (exemplary protein sequence comprises UniProtkB ID No. Q99795), FLT-3 (exemplary protein sequence comprises UniProtkB ID No. P36888), gpNMB (exemplary protein sequence comprises UniProtkB ID No. Q14956), HPV-16 E6 (exemplary protein sequence comprises UniProtkB ID No. P03126), HPV-16 E7 (exemplary protein sequence comprises UniProtkB ID No. P03129), ITGA2 (exemplary protein sequence comprises UniProtkB ID No. P17301), ITGA3 (exemplary protein sequence comprises UniProtkB ID No. P26006), SLC39A6 (exemplary protein sequence comprises UniProtkB ID No. Q13433), MAGE (exemplary protein sequence comprises UniProtkB ID No. Q9HC15), mesothelin (exemplary protein sequence comprises UniProtkB ID No. Q13421), Muc1 (exemplary protein sequence comprises UniProtkB ID No. P15941), Muc16 (exemplary protein sequence comprises UniProtkB ID No. Q8WX17), NaPi2b (exemplary protein sequence comprises UniProtkB ID No. 095436), Nectin-4 (exemplary protein sequence comprises UniProtkB ID No. Q96918), CDH-3 (exemplary protein sequence comprises UniProtkB ID No. Q8WX17), CDH-17 (exemplary protein sequence comprises UniProtkB ID No. E5RJT3), EPHB2 (exemplary protein sequence comprises UniProtkB ID No. P29323), ITGAV (exemplary protein sequence comprises UniProtkB ID No. P06756), ITGB6 (exemplary protein sequence comprises UniProtkB ID No. P18564), NY-ESO-1 (exemplary protein sequence comprises UniProtkB ID No. P78358), PRLR (exemplary protein sequence comprises UniProtkB ID No. P16471), PSCA (exemplary protein sequence comprises UniProtkB ID No. 043653), PTK7 (exemplary protein sequence comprises UniProtkB ID No. Q13308), ROR1 (exemplary protein sequence comprises UniProtkB ID No. Q01973), SLC44A4 (exemplary protein sequence comprises UniProtkB ID No. Q53GD3), SLITRK5 (exemplary protein sequence comprises UniProtkB ID No. Q81W52), SLITRK6 (exemplary protein sequence comprises UniProtkB ID No. Q9HY7), STEAP1 (exemplary protein sequence comprises UniProtkB ID No. Q9UHE8), TIM1 (exemplary protein sequence comprises UniProtkB ID No. Q96D42), Trop2 (exemplary protein sequence comprises UniProtkB ID No. P09758), or WT1 (exemplary protein sequence comprises UniProtkB ID No. P19544), or any combinations thereof, In some embodiments, the target comprises an immune checkpoint protein. In some embodiments, the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA. In some embodiments, the immune checkpoint protein is at least one of: CD27 (exemplary protein sequence comprises UniProtkB ID No. P26842), CD137 (exemplary protein sequence comprises UniProtkB ID No. Q07011), 2B4 (exemplary protein sequence comprises UniProtkB ID No. Q9bZW8), TIGIT (exemplary protein sequence comprises UniProtkB ID No. Q495A1), CD155 (exemplary protein sequence comprises UniProtkB ID No. P15151), ICOS (exemplary protein sequence comprises UniProtkB ID No. Q9Y6W8), HVEM (exemplary protein sequence comprises UniProtkB ID No. O43557), CD40L (exemplary protein sequence comprises UniProtkB ID No. P29965), LIGHT (exemplary protein sequence comprises UniProtkB ID No. O43557), OX40 (exemplary protein sequence comprises UniProtkB ID No.), DNAM-1 (exemplary protein sequence comprises UniProtkB ID No. Q15762), PD-L1 (exemplary protein sequence comprises UniProtkB ID No. Q9ZQ7), PD1 (exemplary protein sequence comprises UniProtkB ID No. Q15116), PD-L2 (exemplary protein sequence comprises UniProtkB ID No. Q9BQ51), CTLA-4 (exemplary protein sequence comprises UniProtkB ID No. P16410), CD8 (exemplary protein sequence comprises UniProtkB ID No. P10966, P01732), CD40 (exemplary protein sequence comprises UniProtkB ID No. P25942), CEACAM1 (exemplary protein sequence comprises UniProtkB ID No. P13688), CD48 (exemplary protein sequence comprises UniProtkB ID No. P09326), CD70 (exemplary protein sequence comprises UniProtkB ID No. P32970), AA2AR (exemplary protein sequence comprises UniProtkB ID No. P29274), CD39 (exemplary protein sequence comprises UniProtkB ID No. P49961), CD73 (exemplary protein sequence comprises UniProtkB ID No. P21589), B7-H3 (exemplary protein sequence comprises UniProtkB ID No. Q5ZPR3), B7-H4 (exemplary protein sequence comprises UniProtkB ID No. Q7Z7D3), BTLA (exemplary protein sequence comprises UniProtkB ID No. Q76A9), IDO1 (exemplary protein sequence comprises UniProtkB ID No. P14902), IDO2 (exemplary protein sequence comprises UniProtkB ID No. Q6ZQW0), TDO (exemplary protein sequence comprises UniProtkB ID No. P48755), KIR (exemplary protein sequence comprises UniProtkB ID No. Q99706), LAG-3 (exemplary protein sequence comprises UniProtkB ID No. P18627), TIM-3 (also known as HAVCR2, exemplary protein sequence comprises UniProtkB ID No. Q8TDQ0), or VISTA (exemplary protein sequence comprises UniProtkB ID No. Q9D659), or any combinations thereof. In some embodiments, the target comprises an immune cell. In some embodiments, the immune cell comprises a T-cell. In some embodiments, the target comprises CD3. In some embodiments, the target comprises CD3ε. In some embodiments, the first binding domain comprises two or more polypeptides linked by a non-cleavable linker. In some embodiments, the protease cleavage site is recognized by a serine protease, a cysteine protease, an aspartate protease, a threonine protease, a glutamic acid protease, a metalloproteinase, a gelatinase, or a asparagine peptide lyase. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, a cathepsin G, a proteinase 3, a neutrophil serine protease 4, a mast cell chymase, and a mast cell tryptase.

One embodiment provides a polynucleotide encoding the conditionally activated binding protein of any one of above embodiments. One embodiment provides a vector comprising the polynucleotide. One embodiment provides a host cell transformed with the vector. One embodiment provides a pharmaceutical composition comprising (i) the conditionally activated binding protein according to any one of above embodiments, the polynucleotide, the vector, or the host cell and (ii) a pharmaceutically acceptable carrier. One embodiment provides a process for the production of the conditionally activated binding protein according to any one of above embodiments, said process comprising culturing a host transformed or transfected with a vector comprising a nucleic acid sequence. One embodiment provides a method for the treatment or amelioration of a proliferative disease or a tumorous disease, comprising the administration of conditionally activated binding protein according to any one of above embodiments to a subject in need of such a treatment or amelioration. In some embodiments, the subject is a human. In some embodiments, the method further comprises administration of an agent in combination with the conditionally activated binding protein according to any one of above embodiments.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which.

FIG. 1 illustrates an exemplary conditionally activated binding protein, in an inactive form, comprising a first binding domain (an HSA binder) bound to human serum albumin (HSA), a second binding domain (anti-target) that is sterically occluded from binding its target.

FIGS. 2A-2E show exemplary schematic structures of ProDrug molecules. FIG. 2A shows a drug linked to an anti-albumin moiety by a cleavable linker. FIG. 2B shows a drug linked to an albumin-binding peptide motif, wherein the peptide motif is linked to a drug by a cleavable linker. FIG. 2C shows a drug linked to a modified albumin by a cleavable linker. FIG. 2D shows a drug linked to a modified albumin by a linker, wherein the modified albumin includes a protease cleavable site. FIG. 2E shows an activated drug. In each schematic structure (from FIGS. 2A-2D) the drug molecule is sterically occluded by the anti-albumin moiety or the modified albumin from binding its target or from being activated at an undesired site or from binding at non-target sites and thereby creating a drug sink.

FIGS. 3A-3E shows exemplary schematic structure of ProTriTAC molecules. FIG. 3A shows a T cell engager molecule linked to an anti-albumin moiety, by a cleavable linker, to form the ProTriTAC molecule. FIG. 3B shows a T cell engager molecule linked to an albumin-binding peptide motif, wherein the peptide motif is linked to the T cell engager by a cleavable linker, to form the ProTriTAC molecule. FIG. 3C shows T cell engager molecule linked to a modified albumin by a cleavable linker, to form the ProTriTAC molecule. FIG. 3D shows a T cell engager molecule linked to a modified albumin by a linker to form the ProTriTAC molecule, wherein the modified albumin includes a protease cleavable site. FIG. 3E shows an activated form of the ProTriTAC molecule. In each schematic structure (from FIGS. 3A-3D) the ProTriTAC molecule is sterically occluded by the anti-albumin moiety or the modified albumin from binding its target or from being activated at an undesired site or from binding at non-target sites and thereby creating a drug sink.

FIG. 4 shows steric occlusion of EGFR ProTriTAC, using a T cell dependent cell cytotoxicity assay.

FIG. 5 shows steric occlusion of EGFR ProTriTAC, using an ELISA CD3 binding assay.

FIG. 6A-C shows exemplary ProCAR (chimeric antigen receptor) or CAR constructs.

FIG. 7 demonstrates steric blocking of anti-EpCAM sdAb H90 ProCAR-T cell killing activity by HSA when assayed at ratios 10:1, 5:1, 2.5:1, and 1.25:1 CAR-T:Target cells.

FIG. 8 provides exemplary arrangements of various domains of a ProTriTAC molecule of this disclosure.

FIG. 9 provides a possible mode of activation of a ProTriTAC molecule of this disclosure.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

A “single chain Fv” or “scFv”, as used herein, refers to a binding protein in which the variable domains of the heavy chain and of the light chain of a traditional two chain antibody are joined to form one chain. Typically, a linker peptide is inserted between the two chains to allow for proper folding and creation of an active binding site.

A “cleavage site for a protease,” or “protease cleavage site,” as meant herein, is an amino acid sequence that can be cleaved by a protease, such as, for example, a matrix metalloproteinase or a furin. Examples of such sites include Gly-Pro-Leu-Gly-Ile-Ala-Gly-Gln or Ala-Val-Arg-Trp-Leu-Leu-Thr-Ala, which can be cleaved by metalloproteinases, and Arg-Arg-Arg-Arg-Arg-Arg, which is cleaved by a furin. In therapeutic applications, the protease cleavage site can be cleaved by a protease that is produced by target cells, for example cancer cells or infected cells, or pathogens.

A “ProTriTAC molecule,” as used herein, refers to a trispecific molecule comprising a conditionally activated binding protein as described herein (comprising a first binding domain that is capable of binding a bulk serum protein and a second binding domain that is sterically occluded from binding a target when the ProTriTAC molecule is in it's activatable form), and a third domain specific for CD3. Upon cleavage of the cleavable linker, the ProTriTAC molecule is activated.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e., after drug absorption and distribution are complete). The half-time for drug elimination can be accurately determined from such a graph.

A “therapeutic agent,” as used herein, includes a “binding molecule.”

The term “binding molecule,” or a “binding domain,” as used interchangeably herein is any molecule, or portion or fragment thereof, or a variant thereof, that can bind to another molecule, cell, complex and/or tissue, and which includes proteins, nucleic acids, carbohydrates, lipids, low molecular weight compounds, and fragments thereof, each having the ability to bind to one or more of a soluble protein, a cell surface protein, a cell surface receptor protein, an intracellular protein, a carbohydrate, a nucleic acid, a hormone, or a low molecular weight compound (small molecule drug), a portion or fragment thereof, or a variant thereof. The binding domains, in some instances, are proteins belonging to the immunoglobulin superfamily, or a non-immunoglobulin molecule.

The term “proteins belonging to immunoglobulin superfamily,” or “immunoglobulin molecules,” as used herein, include proteins that comprise an immunoglobulin fold, such as antibodies and target antigen binding fragments thereof, antigen receptors, antigen presenting molecules, receptors on natural killer cells, antigen receptor accessory molecules, receptors on leukocytes, IgSF cellular adhesion molecules, growth factor receptors, and receptor tyrosine kinases/phosphatases.

The term “antibodies” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, bi-specific antibodies, multi-specific antibodies, and fusion proteins comprising an antigen-binding (also referred to herein as antigen binding) portion of an antibody and a non-antibody protein. The antibodies, in some examples, are detectably labeled, e.g., with a radioisotope, an enzyme that generates a detectable product, a fluorescent protein, and the like. The antibodies, in some cases, are further conjugated to other moieties, such as members of specific binding pairs, e.g., biotin (member of biotin-avidin specific binding pair), and the like. The antibodies, in some cases, are bound to a solid support, including, but not limited to, polystyrene plates or beads, and the like. Also encompassed by the term are Fab′, Fv, F(ab′)2, and or other antigen binding fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody, in some instances, is monovalent or bivalent. An antibody, in some instances, is an Ig monomer, which is a “Y-shaped” molecule that consists of four polypeptide chains: two heavy chains and two light chains connected by disulfide bonds.

The term “non-immunoglobulin molecules,” as used herein, include a growth factor, a hormone, a signaling protein, an inflammatory mediator, ligand, a receptor, or a fragment thereof, a native hormone or a variant thereof being able to bind to its natural receptor; a nucleic acid or polynucleotide sequence being able to bind to complementary sequence or a soluble cell surface or intracellular nucleic acid/polynucleotide binding proteins, a carbohydrate binding moiety being able to bind to other carbohydrate binding moieties, cell surface or intracellular proteins, a low molecular weight compound (drug) that binds to a soluble or cell surface or intracellular target protein. The non-immunoglobulin moleculenon-immunoglobulin moleculeimmunoglobulin molecules, in some cases, include coagulation factors, plasma proteins, fusion proteins, and imaging agents. The non-immunoglobulin molecules do not include a cytokine.

A “cytokine,” as meant herein, refers to intercellular signaling molecules, and active fragments and portions thereof, which are involved in the regulation of mammalian somatic cells. A number of families of cytokines, for example, interleukins, interferons, and transforming growth factors are included.

“Target antigen binding domain”, as used herein, refers to a region which targets a specific antigen. A target antigen binding domain or molecules comprises, for example an sdAb, a scFv, a variable heavy domain (VHH), a full length antibody, or any other peptide that has a binding affinity towards a specific antigen.

Conditionally Active Binding Proteins Comprising a Binding Domain that is Sterically Occluded from Binding its Target

Provided herein, in one embodiment, is conditionally activated binding protein that includes a first binding domain, a second binding domain, a cleavable linker that connects the first and the second binding domains, and is capable of being activated from an inactive form upon cleavage of the linker, for example in a protease rich environment, such as a tumor microenvironment. In the inactive form (also referred to herein as the masked form), the first binding domain is bound to a bulk serum protein and through its binding to the bulk serum protein the first binding domain sterically occludes the second binding domain from binding its target. In some embodiments, the sterical occlusion is due to the close proximity between the bulk serum protein and the second binding domain, in the inactive form of the conditionally activated binding protein.

In some cases, the first binding domain comprises a binding site for a bulk serum protein. In some embodiments, the CDRs within the first binding domains provide a binding site for the bulk serum protein. The bulk serum protein is, in some examples, a globulin, albumin, transferrin, IgG1, IgG2, IgG4, IgG3, IgA monomer, Factor XIII, Fibrinogen, IgE, or pentameric IgM. In some embodiments, the bulk serum protein comprises albumin, fibrinogen, or a globulin.

In some embodiments, the first binding domain comprises a binding site for an immunoglobulin light chain. In some embodiments, the CDRs provide a binding site for the immunoglobulin light chain. The immunoglobulin light chain is, in some examples, an Igκ free light chain or an Igλ free light chain. Variants or fragments of bulk serum proteins exemplified above are also included in this disclosure. A variant of a bulk serum protein, in some embodiments, comprises one or more amino acid substitutions relative to the native sequence of the bulk serum protein. In some examples, the first binding domain comprises any type of binding domain, including but not limited to, domains from a monoclonal antibody, a polyclonal antibody, a recombinant antibody, a human antibody, a humanized antibody. In some embodiments, the first binding domain is a single chain variable fragment (scFv), single-domain antibody such as a heavy chain variable domain (VH), a light chain variable domain (VL), and a variable heavy chain only domain (VHH), for example, of a camelid derived nanobody. In other embodiments, the first binding domain is a non-Ig binding domain, an antibody mimetic, such as anticalins, affilins, affibody molecules, affimers, affitins, alphabodies, avimers, DARPins, fynomers, kunitz domain peptides, and monobodies. In some embodiments, the first binding domain is an engineered scaffold. The engineered scaffold comprises, for example, at least one of: an sdAb, an scFv, an Fab, a VHH, a IgNAR, a VH, a VL, a fibronectin type III domain, an immunoglobulin-like scaffold, a bacterial albumin-binding domain, an adnectin, a monobody, an affibody, an affilin, an affimer, an affitin, an alphabody, an anticalin, an avimer, a centyrin, a DARPin, a cystine knot peptide, a lipocalin, a three-helix bundle scaffold, a protein G-related albumin-binding module, a DNA or RNA aptamer scaffold, or any combinations thereof.

In one embodiment, the bulk serum protein is human serum albumin and the first domain is a human serum albumin binding domain (also referred to herein as an HSA-binder, or an anti-ALB domain, or an anti-albumin binding domain, or an anti-albumin domain). Human serum albumin (HSA) (molecular mass ˜67 kDa) is the most abundant protein in plasma, present at about 50 mg/ml (600 μM) and has a half-life of around 20 days in humans. HSA serves to maintain plasma pH, contributes to colloidal blood pressure, functions as carrier of many metabolites and fatty acids, and serves as a major drug transport protein in plasma. In some cases, the HSA binder is a variant HSA binder and comprises a sequence that has one or more amino acid substitutions relative to wild type HSA-binder sequence (disclosed herein as SEQ ID NO: 43). In some embodiments, the HSA binder is a single domain antibody. In some embodiments, the HSA-binder comprises a sequence as set forth in any one of SEQ ID Nos.: 44-52. Further variants of the foregoing sequences are also included in certain embodiments of this disclosure, such as variants that comprise one or more conservative or non-conservative amino acid substitutions relative to one or more of SEQ ID Nos. 1-10. In some embodiments, the first binding domain comprises a molecular weight of about 5 kDa to about 10 kDa, about 7 kDa to about 15 kDa, about 12 kDa to about 20 kDa, about 16 kDa to about 25 kDa, or more. In certain instances, the first binding domain comprises a molecular weight of about 5 kDa or less if it is a peptide or small molecule entity. In some embodiments, non-covalent association between the first binding domain and a bulk serum protein, such as HSA, extends the elimination half-time of the conditionally activated binding proteins of this disclosure, until the time when it is activated by cleavage of the linker. Following cleavage of the linker, the binding protein is activated and separated from the first binding domain, such as the HSA-binder, and the bulk serum protein. This terminates the half-life extended status of the binding protein and it is capable of being rapidly cleared from the system, as discussed further in the subsequent sections. In addition, the conditionally activated binding protein of this disclosure, in some cases, comprises a “biobetter” version of a biologic. Generally, preparing a biobetter form of a molecule, e.g., an antibody or an antigen binding fragment thereof, involves taking the originator molecule and making specific alterations in it to improve its parameters and thereby make it a more efficacious, less frequently dosed, better targeted, a better tolerated drug, a combination thereof. Thus, a target antigen binding domain masked by an HSA binder which is bound to a half-life extending protein, and conditionally activated in a tumor microenvironment by cleavage of the cleavable linker, gives the target antigen binding domain a significantly longer serum half-life and reduces the likelihood of its undesirable activation in circulation, thereby producing a “biobetter” version of the target antigen binding domain. Similarly, the conditionally activated binding proteins, in some cases, comprise biobetter versions of a non-immunoglobulin molecule. Accordingly, in various embodiments, biobetter versions of immunoglobulin molecules, or the non-immunoglobulin molecules are provided, wherein the biobetter function is attributed to the first binding domain which is capable of sterically occluding the second binding domain, through its binding to a bulk serum protein, such as HSA.

The cleavable linker, for example, comprises a protease cleavage site or a pH dependent cleavage site. The cleavable linker, in certain instances, is cleaved only in a tumor microenvironment. In some examples, the conditionally activated binding protein, in the inactive form when the first binding domain is bound to the bulk serum protein and the linker is not cleaved, the second binding domain, such as a target antigen binding domain, in maintained in an inert state in circulation until the cleavable linker is cleaved off in a tumor microenvironment. The half-life of the target antigen binding domain, such as an antibody or an antigen binding fragment thereof, is thus extended in systemic circulation when it is part of the conditionally activated binding protein. In the inactive form, the binding protein acts as a safety switch that keeps the target antigen binding moiety in an inert state until it reaches the tumor microenvironment where it is conditionally activated by cleavage of the linker and is able to bind its target antigen. The safety switch described above provides several advantages, some examples including (i) expanding the therapeutic window of an immunoglobulin molecule, such as a target antigen binding domain, or a non-immunoglobulin molecule; (ii) reducing target-mediated drug disposition by maintaining the immunoglobulin molecule, such as a target antigen binding domain, or the non-immunoglobulin molecule in an inert state when a conditionally activated protein as described herein is in systemic circulation; (iii) reducing the concentration of undesirably activated proteins in systemic circulation, thereby minimizing the spread of chemistry, manufacturing, and controls related impurities, e.g., pre-activated drug product, endogenous viruses, host-cell proteins, DNA, leachables, anti-foam, antibiotics, toxins, solvents, heavy metals; (iv) reducing the concentration of undesirably activated proteins in systemic circulation, thereby minimizing the spread of product related impurities, aggregates, breakdown products, product variants due to: oxidation, deamidation, denaturation, loss of C-term Lys in MAbs; (v) preventing aberrant activation of an immunoglobulin molecule, such as a target antigen binding domain, or a non-immunoglobulin molecule in circulation; (vi) reducing the toxicities associated with the leakage of activated species from diseased tissue or other pathophysiological conditions, e.g., tumors, autoimmune diseases, inflammations, viral infections, tissue remodeling events (such as myocardial infarction, skin wound healing), or external injury (such as X-ray, CT scan, UV exposure); and (vii) reducing non-specific binding of an immunoglobulin molecule, such as a target antigen binding domain, or a non-immunoglobulin molecule, by enabling rapid clearance of the molecules after they are separated from the safety switch which provided extended half-life.

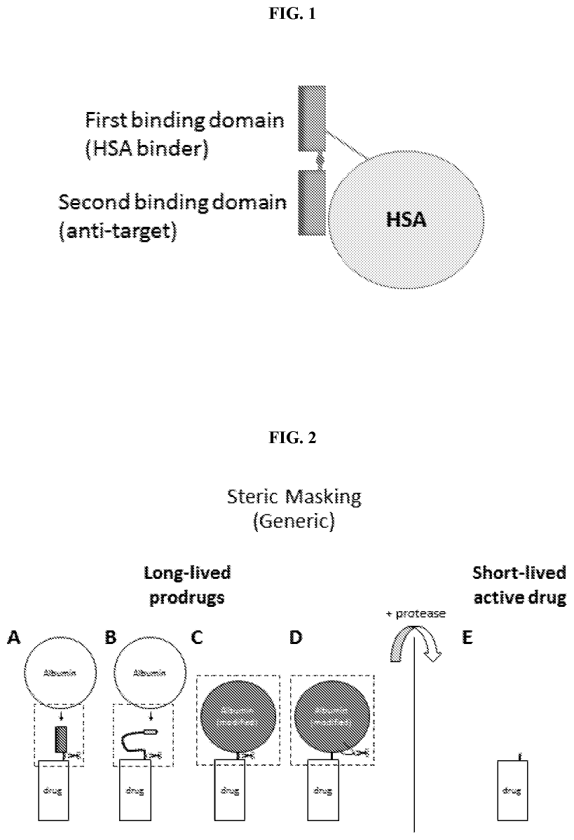

Examples of the second domain, such as a target antigen binding domain, includes, but are not limited to, a T cell engager, a bispecific T cell engager, a dual-affinity re-targeting antibody, a variable heavy domain (VH), a variable light domain (VL), a scFv comprising a VH and a VL domain, a single domain antibody (sdAb), or a variable domain of camelid derived nanobody (VHH), a non-Ig binding domain, i.e., antibody mimetic, such as anticalins, affilins, affibody molecules, affimers, affitins, alphabodies, avimers, DARPins, fynomers, kunitz domain peptides, and monobodies, a ligand or peptide. In some examples, the target antigen binding domain is a VHH domain. In some examples, the target antigen binding domain is a sdAb. In some instances, the target antigen binding domain is specific for a tumor antigen or for CD3ε. The binding of the target antigen binding domain to its target, e.g., a tumor antigen such as EGFR, is masked by the HSA binder. One exemplary conditionally activated binding protein is shown in FIG. 1.

In another embodiment the conditionally activated binding protein comprises the first binding domain masks the interaction between a non-immunoglobulin molecule and its target or binding partner, that is, the second binding domain is a non-immunoglobulin molecule. Examples of non-immunoglobulin molecules include, but are not limited to, a growth factor, a hormone, a signaling protein, an inflammatory mediator, ligand, a receptor, or a fragment thereof, a native hormone or a variant thereof being able to bind to its natural receptor; a nucleic acid or polynucleotide sequence being able to bind to complementary sequence or a soluble cell surface or intracellular nucleic acid/polynucleotide binding proteins, a carbohydrate binding moiety being able to bind to other carbohydrate binding moieties, cell surface or intracellular proteins, a low molecular weight compound (drug) that binds to a soluble or cell surface or intracellular target protein. The non-immunoglobulin binding molecules, in some cases, include coagulation factors, plasma proteins, fusion proteins, and imaging agents. The non-immunoglobulin molecules do not include a cytokine.

The protease cleavable linker, in some cases, enables activation of a prodrug/ProTriTAC molecule comprising a conditionally activated binding protein of this disclosure, in a single proteolytic event, thereby allowing more efficient conversion of the prodrug/ProTriTAC molecule in tumor microenvironment. Further, tumor-associated proteolytic activation, in some cases, reveals active T cell engager (such as a ProTriTAC molecule comprising a conditionally activated binding protein of this disclosure, and a CD3 binding domain) with minimal off-tumor activity after activation. The present disclosure, in some embodiments, provides a half-life extended T cell engager format (ProTriTAC) comprising a conditionally activated binding protein of this disclosure, which in some cases represents a new and improved approach to engineer conditionally active T cell engagers.

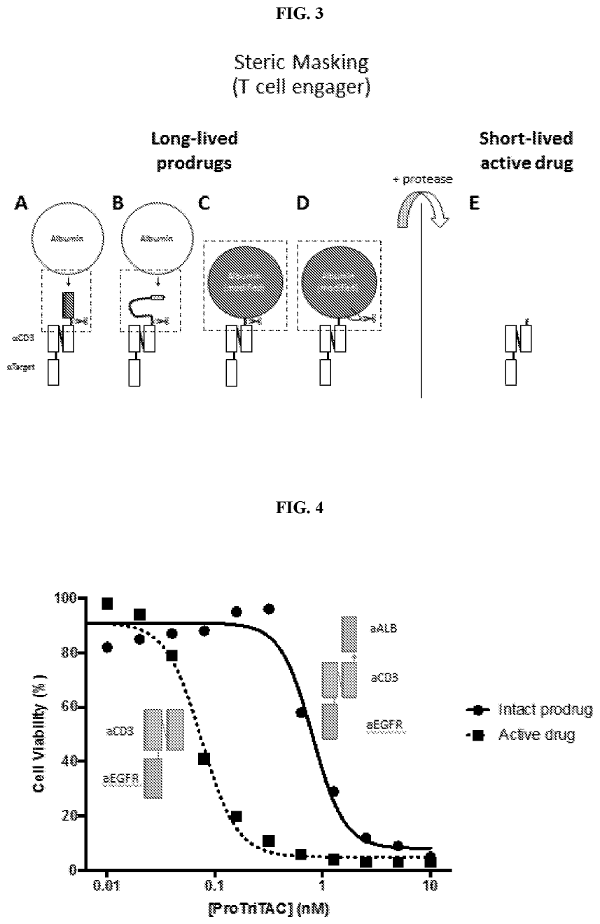

Steric masking of a T cell engager, such as the ProTriTAC molecule is shown in FIG. 3, with a possible mode of action of the same. FIG. 2 provides an exemplary schematic steric masking of drug, with a possible mode of action of the same.

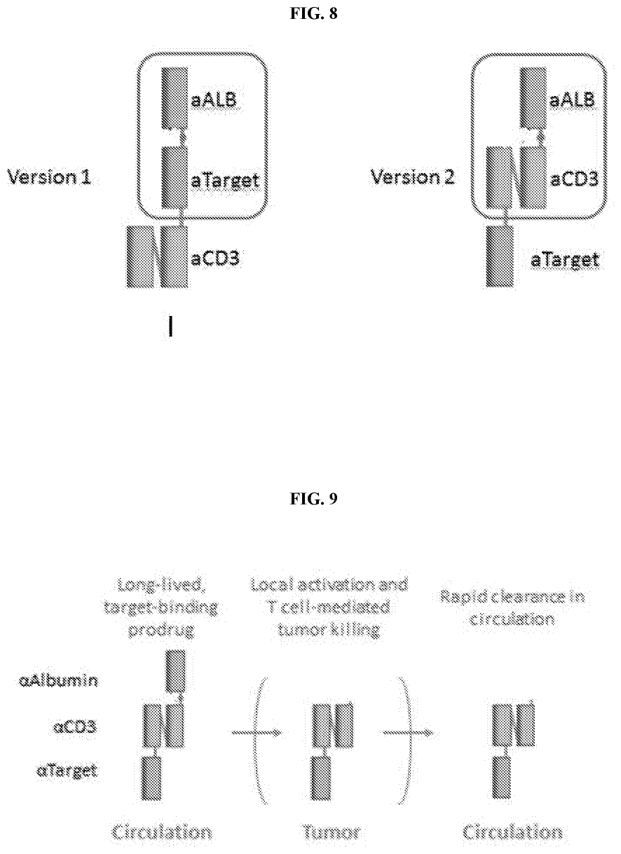

An exemplary trispecific molecule of this disclosure contains a first domain that comprises an anti-albumin domain tethered to a cleavable linker, the second domain is an anti-target domain (such as, a domain specific for a tumor antigen); and a third domain that is an anti-CD3 binding domain. The anti-albumin domain, in some embodiments, comprises a CDR loop specific for binding albumin and when the anti-albumin domain is bound to a bulk serum protein (such as albumin) the anti-target domain or the anti-CD3 domain are sterically occluded from binding their target. Two configurations of a ProTriTAC molecule are shown in FIG. 8. FIG. 9 shows a possible mode of activation of a ProTriTAC molecule.

In some embodiments, the conditionally activated binding protein is less than about 80 kDa. In some embodiments, the conditionally activated binding protein is about 50 to about 75 kDa. In some embodiments, the conditionally activated binding protein is less than about 60 kDa.

In some embodiments of this disclosure are provided conditionally activated chimeric antigen receptors that comprise an antiCD3 domain, an anti-target domain (target can be any of the antigens described above), and an anti-Albumin domain. In some embodiments, the target binding domains or the CD3 binding domains of the conditionally activated CARs are sterically occluded from binding their targets by the anti-albumin domain, in the presence of human serum albumin.

### Targets of Conditionally Activated Binding Proteins

The conditionally activated binding proteins described herein are activated by cleavage of the at least one cleavable linker attached to the first binding domain within said conditionally activated proteins. It is contemplated that in some cases the activated binding protein binds to a target antigen involved in and/or associated with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated binding proteins disclosed herein. Target antigens, in some cases, are expressed on the surface of a diseased cell or tissue, for example a tumor or a cancer cell. Target antigens include but are not limited to EpCAM, EGFR, HER-2, HER-3, c-Met, FoIR, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, or WT1. In some embodiments, the target antigen is an immune checkpoint protein. Examples of immune checkpoint proteins include but are not limited to CD27, CD137, 2B4, TIGIT, CD155, ICOS, HVEM, CD40L, LIGHT, TIM-1, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDOL IDO2, TDO, KIR, LAG-3, TIM-3, or VISTA. In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, inflammed or fibrotic tissue cell. In some instances, the second binding domain is capable of binding a CD3 binding domain. In some instances, the second binding domain is capable of binding a CD3ε binding domain.

### Cleavable Linkers and Protease Sites

It is contemplated herein that the conditionally activated binding proteins described herein comprise at least one cleavable linker. In one aspect, the cleavable linker comprises a polypeptide having a sequence recognized and cleaved in a sequence-specific manner. The cleavage, in certain examples, is enzymatic, based on pH sensitivity of the cleavable linker, or by chemical degradation. A protease cleavable linker, in some cases, is recognized in a sequence-specific manner by a matrix metalloprotease (MMP), for example a MMP9. In some cases, the protease cleavable linker is recognized by a MMP9 comprises a polypeptide having an amino acid sequence PR(S/T)(L/I)(S/T). In some cases, the protease cleavable linker is recognized by a MMP9 and comprises a polypeptide having an amino acid sequence LEATA. In some cases, the protease cleavable linker is recognized in a sequence-specific manner by MMP11. In some cases, the protease cleavable linker recognized by MMP11 comprises a polypeptide having an amino acid sequence GGAANLVRGG (SEQ IN NO: 3). In some cases, the protease cleavable linker is recognized by a protease disclosed in Table 1. In some cases, the protease cleavable linker is recognized by a protease disclosed in Table 1 comprises a polypeptide having an amino acid sequence selected from a sequence disclosed in Table 1 (SEQ ID NOS: 1-42).

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP7, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, type II transmembrane serine proteases (TTSP), neutrophil serine protease, cathepsin G, proteinase 3, neutrophil serine protease 4, mast cell chymase, and mast cell tryptases.

TABLE 1
Exemplary Proteases and Protease Recognition Sequences,
Contained within Exemplary First Binding
Domains of this disclosure

| | Cleavage Domain | |
|---|---|---|
| Protease | Sequence | SEQ ID NO: |
| MMP7 | KRALGLPG | 1 |
| MMP7 | (DE)₈RPLALWRS(DR)₈ | 2 |
| MMP9 | PR(S/T)(L/I)(S/T) | 3 |
| MMP9 | LEATA | 4 |
| MMP11 | GGAANLVRGG | 5 |
| MMP14 | SGRIGFLRTA | 6 |
| MMP | PLGLAG | 7 |
| MMP | PLGLAX | 8 |
| MMP | PLGC(me)AG | 9 |
| MMP | ESPAYYTA | 10 |
| MMP | RLQLKL | 11 |
| MMP | RLQLKAC | 12 |
| MMP2, MMP9, MMP14 | EP(Cit)G(Hof)YL | 13 |
| Urokinase plasminogen | SGRSA | 14 |
| activator (uPA) | | |
| Urokinase plasminogen | DAFK | 15 |
| activator (uPA) | | |
| Urokinase plasminogen | GGGRR | 16 |
| activator (uPA) | | |
| Lysosomal Enzyme | GFLG | 17 |
| Lysosomal Enzyme | ALAL | 18 |
| Lysosomal Enzyme | FK | 19 |
| Cathepsin B | NLL | 20 |
| Cathepsin D | PIC(Et)FF | 21 |
| Cathepsin K | GGPRGLPG | 22 |
| Prostate Specific Antigen | HSSKLQ | 23 |
| Prostate Specific Antigen | HSSKLQL | 24 |
| Prostate Specific Antigen | HSSKLQEDA | 25 |
| Herpes Simplex Virus | LVLASSSFGY | 26 |
| Protease | | |
| HIV Protease | GVSQNYPIVG | 27 |
| CMV Protease | GVVQASCRLA | 28 |
| Thrombin | F(Pip)RS | 29 |
| Thrombin | DPRSFL | 30 |
| Thrombin | PPRSFL | 31 |
| Caspase-3 | DEVD | 32 |
| Caspase-3 | DEVDP | 33 |
| Caspase-3 | KGSGDVEG | 34 |
| Interleukin 1β | GWEHDG | 35 |
| converting enzyme | | |
| Enterokinase | EDDDDKA | 36 |
| FAP | KQEQNPGST | 37 |
| Kallikrein 2 | GKAFRR | 38 |
| Plasmin | DAFK | 39 |
| Plasmin | DVLK | 40 |
| Plasmin | DAFK | 41 |
| TOP | ALLLALL | 42 |
| MMP9 + matriptase | KPLGLQARVV | 62 |
| MMP9 + matriptase + uPA | PQASTGRSGG | 63 |
| MMP9 + matriptase + uPA | PQGSTGRAAG | 64 |
| Matriptase + uPA | PPASSGRAGG | 65 |
| MMP9 + matriptase | PIPVQGRAH | 66 |
| MMP9 + matriptase + uPA | PQGSTARSAG | 67 |

Proteases are known to be secreted by some diseased cells and tissues, for example tumor or cancer cells, creating a microenvironment that is rich in proteases or a protease-rich microenvironment. In some case, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen binding proteins to have additional specificity because T cells will not be bound by the antigen binding protein except in the protease rich microenvironment of the targeted cells or tissue. The first binding domain attached to the cleavable linker and bound to a bulk serum protein thus sterically occludes the binding of the second binding domain to its target(s).

### Protein Variants

In certain embodiments, amino acid sequence variants of the conditionally activated binding proteins described herein are contemplated. For example, in certain embodiments amino acid sequence variants of the first binding domain or any other domains within the conditionally activated binding proteins described herein are contemplated to improve the binding affinity alone or along with other biological properties of the binding proteins. Exemplary method for preparing such amino acid variants include, but are not limited to, introducing appropriate modifications into the nucleotide sequences encoding the binding proteins, or by peptide synthesis. Such modifications include, for example, deletions from, and/or insertions into and/or substitutions of residues within the amino acid sequences of the binding moieties.

Any combination of deletion, insertion, and substitution are made, in various embodiments, to the conditionally activated binding proteins described herein, to arrive at the final construct, provided that the final construct possesses a desired characteristic, e.g., capability of the first domain to bind a bulk serum protein, that of the second binding domain to bind its target, once sterical occlusion is removed. In certain embodiments, variants having one or more amino acid substitutions are provided. In some cases, sites of interest for substitution mutagenesis include the CDRs and the framework regions of the first and second binding domains. Amino acid substitutions are introduced, desired activity, e.g., retained/improved bulk serum protein/target antigen binding, decreased immunogenicity, or improved antibody-dependent cell mediated cytotoxicity (ADCC) or complement dependent cytotoxicity (CDC). Both conservative and non-conservative amino acid substitutions are contemplated for preparing the protein variants. Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the conditionally activated binding proteins are isolated by screening combinatorial libraries, for example, by generating phage display libraries and screening such libraries for binding proteins possessing the desired binding characteristics towards a target, such as a tumor antigen expressed on a cell surface, or a bulk serum protein.

In another example of a substitution to create a variant, one or more hypervariable region residues of a parent binding protein are substituted. In general, variants are then selected based on improvements in desired properties compared to a parent binding protein, for example, increased affinity, reduced affinity, reduced immunogenicity, increased pH dependence of binding. For example, an affinity matured variant conditionally activated binding protein is generated, in some cases, e.g., using phage display-based affinity maturation techniques.

In some cases, substitutions are made in hypervariable regions (HVR) of an immunoglobulin molecule within the conditionally activated binding protein to generate variants and then selected based on binding affinity to a target antigen binding domain, to a half-life extending domain, or both, i.e., by affinity maturation. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. Substitutions can be in one, two, three, four, or more sites within a parent antibody sequence.

In some embodiments, the conditionally activated binding protein as described herein, is “humanized”, or “camelized,” i.e., by replacing one or more amino acid residues in the amino acid sequence of a naturally occurring immunoglobulin molecule (and in particular in the framework sequences) by one or more of the amino acid residues that occur at the corresponding position(s) in an equivalent binding moiety from a conventional 4-chain antibody from a human being, or a camelid species.

### Modifications

The conditionally activated binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the conditionally activated binding proteins described herein, including the peptide backbone, the amino acid side-chains, and the amino or carboxyl termini. Certain common peptide modifications that are useful for modification of the conditionally activated binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, the conditionally activated binding proteins of the disclosure are capable of being conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc.

### Polynucleotides

Also provided, in some embodiments, are polynucleotide molecules encoding the conditionally activated binding proteins as described herein, or various domains within the proteins. In some embodiments, the polynucleotide molecules are provided as a DNA construct. In other embodiments, the polynucleotide molecules are provided as a messenger RNA transcript.

The polynucleotide molecules are constructed by methods such as by combining the genes encoding the various binding domains within the conditionally activated binding proteins of this disclosure. Each binding domain in some cases comprises a single polypeptide or in some cases comprises two or more polypeptides separated by peptide linkers or, in other embodiments, two or more polypeptides directly linked by a peptide bond. In some embodiments, the polynucleotides for the various domains are formed into a single genetic construct operably linked to a suitable promoter, and optionally a suitable transcription terminator, and expressed it in bacteria or other appropriate expression system such as, for example CHO cells. Depending on the vector system and host utilized, any number of suitable transcription and translation elements, including constitutive and conditionally activated promoters, are used. In some examples, the promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described conditionally activated binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the conditionally activated binding proteins as described herein, in some embodiments, are produced by introducing a vector encoding the moiety or the protein as described above into a host cell and culturing said host cell under conditions whereby the moiety or the protein domains are expressed.

### Pharmaceutical Compositions

Also provided, in some embodiments, are pharmaceutical compositions comprising a therapeutically effective amount of a conditionally activated binding protein of the present disclosure, and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The conditionally activated binding proteins described herein are contemplated for use as a medicament. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

In some embodiments, the conditionally activated binding proteins of this disclosure are administered at a dosage of up to 10 mg/kg at a frequency of once a week. In some cases, the dosage ranges from about 1 ng/kg to about 10 mg/kg. In some embodiments, the dose is from about 1 ng/kg to about 10 ng/kg, about 5 ng/kg to about 15 ng/kg, about 12 ng/kg to about 20 ng/kg, about 18 ng/kg to about 30 ng/kg, about 25 ng/kg to about 50 ng/kg, about 35 ng/kg to about 60 ng/kg, about 45 ng/kg to about 70 ng/kg, about 65 ng/kg to about 85 ng/kg, about 80 ng/kg to about 1 μg/kg, about 0.5 μg/kg to about 5 μg/kg, about 2 μg/kg to about 10 μg/kg, about 7 μg/kg to about 15 μg/kg, about 12 μg/kg to about 25 μg/kg, about 20 μg/kg to about 50 μg/kg, about 35 μg/kg to about 70 μg/kg, about 45 μg/kg to about 80 μg/kg, about 65 μg/kg to about 90 μg/kg, about 85 μg/kg to about 0.1 mg/kg, about 0.095 mg/kg to about 10 mg/kg. In some cases, the dosage is about 0.1 mg/kg to about 0.2 mg/kg; about 0.25 mg/kg to about 0.5 mg/kg, about 0.45 mg/kg to about 1 mg/kg, about 0.75 mg/kg to about 3 mg/kg, about 2.5 mg/kg to about 4 mg/kg, about 3.5 mg/kg to about 5 mg/kg, about 4.5 mg/kg to about 6 mg/kg, about 5.5 mg/kg to about 7 mg/kg, about 6.5 mg/kg to about 8 mg/kg, about 7.5 mg/kg to about 9 mg/kg, or about 8.5 mg/kg to about 10 mg/kg. The frequency of administration, in some embodiments, is about less than daily, every other day, less than once a day, twice a week, weekly, once in 7 days, once in two weeks, once in two weeks, once in three weeks, once in four weeks, or once a month. In some cases, the frequency of administration is weekly. In some cases, the frequency of administration is weekly and the dosage is up to 10 mg/kg. In some cases, duration of administration is from about 1 day to about 4 weeks or longer.

### Methods of Treatment

Also provided herein, in some embodiments, are methods and uses for stimulating the immune system of an individual in need thereof comprising administration of a conditionally activated binding protein as described herein. In some instances, administration induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue. In some embodiments, the target antigen is an immune checkpoint protein.

Also provided herein are methods and uses for a treatment of a disease, disorder or condition associated with a target antigen comprising administering to an individual in need thereof a conditionally activated binding protein as described herein, which comprises a first binding domain that is capable of binding a half-life extending protein masking a second binding domain from binding its target, by steric occlusion through binding between the first binding domain and the half-life extending protein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an auto-immune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a melanoma. In a further instance, the cancer is non-small cell lung cancer. In yet further instance, the cancer is breast cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the conditionally activated binding proteins as described herein, which comprises a first binding domain that is capable of binding a half-life extending protein masking a second binding domain from binding its target, by steric occlusion through binding between the first binding domain and the half-life extending protein is administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the conditionally activated binding proteins described herein are administered in combination with anti-diarrheal agents, anti-emetic agents, analgesics, opioids and/or non-steroidal anti-inflammatory agents. In some embodiments, the conditionally activated binding proteins as described herein are administered before, during, or after surgery.

SEQUENCE TABLE

| | | |
|---|---|---|
| 43 | wt anti-HSA | EVQLVESGGGLVQPGNSLRLSCAASGFTFSSFGMSWVRQAPGKGLEWVSS |
| | | ISGSGSDTLYADSVKGRFTISRDNAKTTLYLQMNSLRPEDTAVYYCTIGG |
| | | SLSRSSQGTLVTVSS |
| 44 | Anti-HSA sdAb clone | EVQLVESGGGLVQPGNSLRLSCAASGFTFSRFGMSWVRQAPGKGLEWVSS |
| | 6C | ISGSGSDTLYADSVKGRFTISRDNAKTTLYLQMNSLRPEDTAVYYCTIGG |
| | | SLSRSSQGTLVTVSS |
| 45 | Anti-HSA sdAb clone | EVQLVESGGGLVQPGNSLRLSCAASGFTFSKFGMSWVRQAPGKGLEWVSS |
| | 7A | ISGSGADTLYADSLKGRFTISRDNAKTTLYLQMNSLRPEDTAVYYCTIGG |
| | | SLSKSSQGTLVTVSS |
| 46 | Anti-HSA sdAb clone | EVQLVESGGGLVQPGNSLRLSCAASGFTYSSEGMSWVRQAPGKGLEWVSS |
| | 7G | ISGSGSDTLYADSVKGRFTISRDNAKTTLYLQMNSLRPEDTAVYYCTIGG |
| | | SLSKSSQGTLVTVSS |
| 47 | Anti-HSA sdAb clone | EVQLVESGGGLVQPGNSLRLSCAASGFTFSKFGMSWVRQAPGKGLEWVSS |
| | 8H | ISGSGTDTLYADSVKGRFTISRDNAKTTLYLQMNSLRPEDTAVYYCTIGG |
| | | SLSRSSQGTLVTVSS |
| 48 | Anti-HSA sdAb clone | EVQLVESGGGLVQPGNSLRLSCAASGFTFSRFGMSWVRQAPGKGLEWVSS |
| | 9A | ISGSGSDTLYADSVKGRFTISRDNAKTTLYLQMNSLRPEDTAVYYCTIGG |
| | | SLSKSSQGTLVTVSS |
| 49 | Anti-HSA sdAb clone | EVQLVESGGGLVQPGNSLRLSCAASGFTFSKFGMSWVRQAPGKGLEWVSS |
| | 10G | ISGSGRDTLYADSVKGRFTISRDNAKTTLYLQMNSLRPEDTAVYYCTIGG |
| | | SLSVSSQGTLVTVSS |
| 50 | Anti-HSA sdAb clone | EVQLVESGGGLVQPGNSLRLSCAASGFTFSRFGMSWVRQAPGKGLEWVSS |
| | 6CE | ISGSGSDTLYAESVKGRFTISRDNAKTTLYLQMNSLRPEDTAVYYCTIGG |
| | | SLSRSSQGTLVTVSS |
| 51 | Anti-HSA sdAb clone | EVQLVESGGGLVQPGNSLRLSCAASGFTFSKFGMSWVRQAPGKGLEWVSS |
| | 8HE | ISGSGTDTLYAESVKGRFTISRDNAKTTLYLQMNSLRPEDTAVYYCTIGG |
| | | SLSRSSQGTLVTVSS |
| 52 | Anti-HSA sdAb clone | EVQLVESGGGLVQPGNSLRLSCAASGFTFSKFGMSWVRQAPGKGLEWVSS |
| | l0GE | ISGSGRDTLYAESVKGRFTISRDNAKTTLYLQMNSLRPEDTAVYYCTIGG |
| | | SLSVSSQGTLVTVSS |
| 53 | C1038 | EVQLVESGGGLVQPGNSLTLSCAASGFTFSKFGMSWVRQAPGKGLEWVSS |
| | 3WT-HL aALB | ISGSGRDTLYADSVKGRFTISRDNAKTTLYLQMNSLRPEDTAVYYCTIGG |
| | | SLSVSSQGTLVTVSS |
| 54 | substrate | GGGGKPLGLQARVVGGGGT |
| 55 | aCD3 (VH-VL) | EVQLVESGGGLVQPGGSLKLSCAASGFTFNKYAINWVRQAPGKGLEWVAR |
| | | IRSKYNNYATYYADQVKDRFTISRDDSKNTAYLQMNNLKTEDTAVYYCVR |
| | | HANFGNSYISYWAYWGQGTLVTVSSGGGGSGGGGSGGGGSQTVVTQEPSL |
| | | TVSPGGTVTLTCASSTGAVTSGNYPNWVQQKPGQAPRGLIGGTKFLVPGT |
| | | PARFSGSLLGGKAALTLSGVQPEDEAEYYCTLWYSNRWVFGGGTKLTVL |
| 56 | linker | GGGGSGGGS |
| 57 | aEGFR (G8) | EVQLVESGGGLVQPGGSLRLSCAASGRTFSSYAMGWFRQAPGKEREFVVA |
| | | INWASGSTYYADSVKGRFTISRDNSKNTLYLQMNSLRAEDTAVYYCAAGY |
| | | QINSGNYNFKDYEYDYWGQGTLVTVSSHHHHHH |
| 58 | aCD3 (VH-VL) | QTVVTQEPSLTVSPGGTVTLTCASSTGAVTSGNYPNWVQQKPGQAPRGLI |
| | | GGTKFLVPGTPARFSGSLLGGKAALTLSGVQPEDEAEYYCTLWYSNRWVF |
| | | GGGTKLTVLGGGGSGGGGSGGGGSEVQLVESGGGLVQPGGSLKLSCAASG |
| | | FTFNKYAINWVRQAPGKGLEWVARIRSKYNNYATYYADQVKDRFTISRDD |
| | | SKNTAYLQMNNLKTEDTAVYYCVRHANFGNSYISYWAYWGQGTLVTVSS |
| 59 | C2483 | MALPVTALLLPLALLLHAARPEVQLLESGGGLVQPGGSLTLSCAASGFIF |
| | | RAASMAWYRQSPGNERELVASISSGAFTNYADSVKARFTISRDNSKNTLY |
| | | LQMNSLRAEDTAVYYCGATFLRSDGHHTINGQGTLVTVSSTSDYKDDDDK |
| | | TTTPAPRPPTPAPTIASQPLSLRPEACRPAAGGAVHTRGLDFACDIYIWA |
| | | PLAGTCGVLLLSLVITLYKRGRKKLLYIFKQPFMRPVQTTQEEDGCSCRF |
| | | PEEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDGLY |
| | | QGLSTATKDTYDALHMQALPPR |
| 60 | C2790 | MALPVTALLLPLALLLHAARPEVQLVESGGGLVQPGNSLRLSCAASGFTF |
| | | SKFGMSWVRQAPGKGLEWVSSISGSGRDTLYADSVKGRFTISRDNAKTTL |
| | | YLQMNSLRPEDTAVYYCTIGGSLSVSSQGTLVTVSSGSGGSGGGGSGGGG |
| | | GSGEVQLLESGGGLVQPGGSLTLSCAASGFIFRAASMAWYRQSPGNEREL |
| | | VASISSGAFTNYADSVKARFTISRDNSKNTLYLQMNSLRAEDTAVYYCGA |
| | | TFLRSDGHHTINGQGTLVTVSSTSDYKDDDDKTTTPAPRPPTPAPTIASQ |
| | | PLSLRPEACRPAAGGAVHTRGLDFACDIYIWAPLAGTCGVLLLSLVITLY |
| | | KRGRKKLLYIFKQPFMRPVQTTQEEDGCSCRFPEEEEGGCELRVKFSRSA |
| | | DAPAYQQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHMQA |
| | | LPPR |
| 61 | C2780 | MALPVTALLLPLALLLHAARPQVQLVESGGALVQPGGSLRLSCAASGFPV |
| | | NRYSMRWYRQAPGKEREWVAGMSSAGDRSSYEDSVKGRFTISRDDARNTV |
| | | YLQMNSLKPEDTAVYYCNVNVGFEYWGQGTQVTVSSTSDYKDDDDKTTTP |
| | | APRPPTPAPTIASQPLSLRPEACRPAAGGAVHTRGLDFACDIYIWAPLAG |
| | | TCGVLLLSLVITLYKRGRKKLLYIFKQPFMRPVQTTQEEDGCSCRFPEEE |
| | | EGGCELRVKFSRSADAPAYQQGQNQLYNELNLGRREEYDVLDKRRGRDPE |
| | | MGGKPRRKNPQEGLYNELQKDKMAEAYSEIGMKGERRRGKGHDGLYQGLS |
| | | TATKDTYDALHMQALPPR |

### EXAMPLES

The examples below further illustrate the described embodiments without limiting the scope of the disclosure.

### Example 1: A Conditionally Activated Binding Protein of this Disclosure Sterically Occludes an EGFR Binding Domain and is Activated Only in Protease Rich Environment

Cells overexpressing EGFR and a matrix metalloprotease are incubated with either (a) an exemplary conditionally activated binding protein according to the present disclosure (comprising a first binding domain that is specific towards HSA and a second binding domain that is specific towards EGFR and a protease cleavable linker) or (b) a control binding protein that has a first and a second binding domain where the first domain is not capable of binding a bulk serum protein and the second binding domain is specific towards EGFR, and a protease cleavable linker.

Results indicate that in case of the control protein which lacks a first binding domain that is capable of binding to a bulk serum protein, the second binding domain is not prevented from binding EGFR. Whereas, in case of the exemplary conditionally activated binding protein, the presence of the first binding domain sterically occludes the second binding domain from binding EGFR, and no EGFR binding is observed.

In a further study, the conditionally activated binding protein is incubated with cells that over express EGFR and a matrix metalloprotease and (b) with cells that overexpress EGFR but do not overexpress a matrix metalloprotease. EGFR binding is observed only in case of the cells that overexpress both EGFR and matrix metalloprotease. No binding is observed in incubating the conditionally activated binding protein with cells that overexpress EGFR but do not overexpress a matrix metalloprotease. Thus, the protease cleavable linker is selectively cleaved in a protease rich environment. Thus, the exemplary conditionally activated binding protein of the present disclosure is advantageous, for example, in terms of reducing off-tumor toxicity.

### Example 2: EGFR ProTriTAC by Steric Occlusion Confers at Least 10× Functional Masking in a T Cell Cytotoxicity Assay

An exemplary EGFR targeting ProTriTAC molecule was used for this study. Various domains of the EGFR targeting ProTriTAC molecule had the following sequences: anti-ALB domain (SEQ ID No. 53), cleavable linker comprising a protease (matriptase) substrate (SEQ ID No. 54), anti-CD3 domain (VH/VL) (SEQ ID No. 55), linker (SEQ ID No. 56), and an anti-EGFR domain (SEQ ID No. 57).

Function of the EGFR targeting ProTriTAC, either intact or matriptase-activated variants, was assessed for their ability to mediate T cell redirected killing of CaOV4 cells in a T cell-dependent cellular cytotoxicity assay. This T cell function assay was performed in the presence (15 mg/ml) of human serum albumin. Results (provided in FIG. 4) show that the anti-albumin domain, binding to serum albumin, on the intact ProTriTAC confers 10× functional masking compared to the active drug moiety of ProTriTAC.

### Example 3: EGFR ProTriTAC Steric Occlusion is Mediated by Binding to Human Serum Albumin in an ELISA Binding Assay

An exemplary EGFR targeting ProTriTAC molecule was used for this study. Various domains of the EGFR targeting ProTriTAC molecule had the following sequences: anti-ALB domain (SEQ ID No. 53), cleavable linker comprising a protease (matriptase) substrate (SEQ ID No. 54), anti-CD3 domain (VH/VL) (SEQ ID No. 58), linker (SEQ ID No. 56), and an anti-EGFR domain (SEQ ID No. 57).

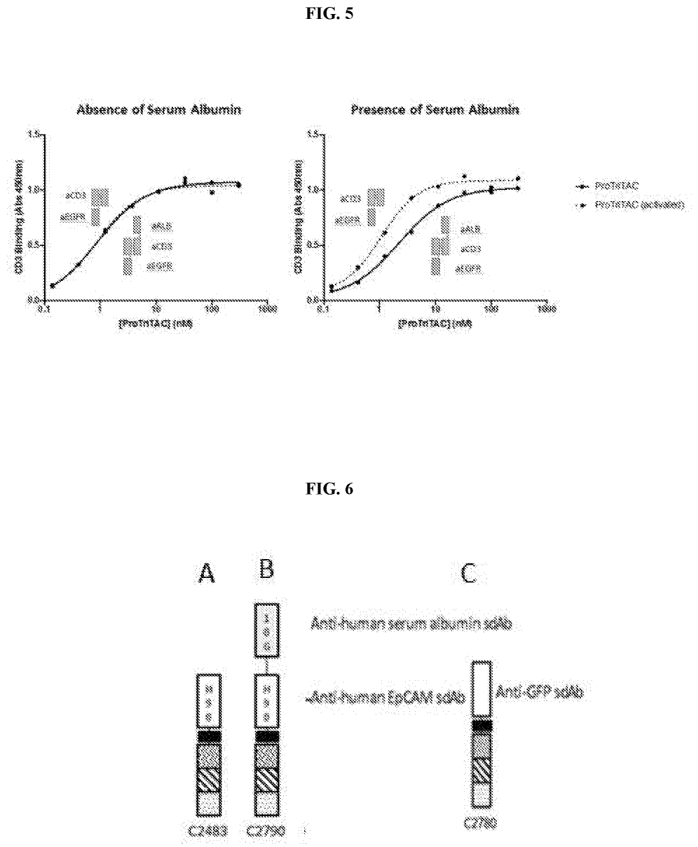

Binding of ProTriTAC, either intact or matriptase-activated variants, was assessed for their ability to bind to human CD3e protein by ELISA. The ELISA assay was performed in the absence or presence (15 mg/ml) of human serum albumin. Results (FIG. 5) show that steric masking of ProTriTAC, in the presence of serum albumin, reduced the binding of ProTriTAC to its target antigen CD3e.

### Example 4: Construction and Testing of Exemplary Multivalent Target Binding Proteins

Constructs

The following ProCAR constructs were made. Construct C2483 includes an anti-human EpCAM sdAb, a FLAG epitope, a CD8 hinge/transmembrane domain, a 4-1BB intracellular domain, and a CD3 zeta intracellular domain (FIG. 6A; SEQ ID NO: 59, H90=anti-EpCAM). Construct C2790 includes an anti-human serum albumin sdAb, an anti-human EpCAM sdAb, a FLAG epitope, a CD8 hinge/transmembrane domain, a 4-1BB intracellular domain, and a CD3 zeta intracellular domain (FIG. 6B; SEQ ID NO: 60, 10G=anti-ALB; H90=anti-EpCAM). FIG. 6C illustrates construct C2780 which includes an anti-GFP sdAb, a FLAG epitope, a CD8 hinge/transmembrane domain, a 4-1BB intracellular domain, and a CD3 zeta intracellular domain (SEQ ID NO: 61).

Steric Blocking by HSA of Anti-EpCAM sdAb H90

300,000 primary human T cells isolated from healthy donors were infected with 1 mL lentiviral supernatant made from the indicated constructs from FIG. 6 to generate anti-EpCAM CAR-T cells, which were subsequently co-cultured at various ratios (CAR-T:Target cells) with EpCAM-expressing cancer cells that stably express luciferase in the presence or absence of human serum albumin (HSA). Luciferase activity was read 72 hours later as a proxy for cancer cell viability and normalized to the anti-GFP control CAR-T cells, C2780.

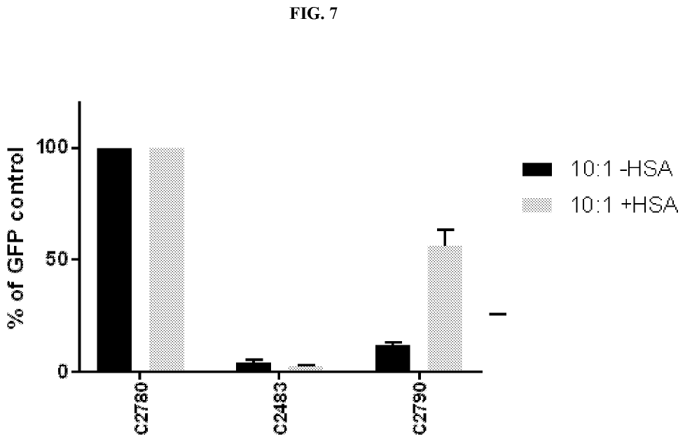

The data provided in FIG. 7 demonstrate steric blocking by HSA of the anti-EpCAM sdAb H90.

What is claimed is:

1. A conditionally activated binding protein, comprising, in an inactive form:
(i) a first binding domain that is capable of binding a bulk serum protein;
(ii) a second binding domain that is sterically occluded from binding a target; and
(iii) a cleavable linker connecting the first and the second binding domains, wherein upon cleavage of the cleavable linker the binding protein is activated and the second binding domain is capable of binding the target.

2. The conditionally activated binding protein of claim 1, wherein the bulk serum protein comprises albumin, transferrin, IgG1, IgG2, IgG4, IgG3, IgA monomer, Factor XIII, Fibrinogen, IgE, pentameric IgM, any variants thereof, any fragments thereof, or a fusion protein comprising any combination thereof.

3. The conditionally activated binding protein of claim 1 or 2, wherein in the inactive form the first binding domain is bound to the bulk serum protein.

4. The conditionally activated binding protein of any one of claims 1-3, wherein in the inactive form the bulk serum protein is in close proximity to the second binding domain, thereby sterically occluding the second binding domain from binding its target.

5. The conditionally activated binding protein of any one of claims 1-4, wherein the first and the second binding domains are connected by a protease cleavable linker.

6. The conditionally activated binding protein of claim 5, wherein the cleavable linker comprises a protease cleavage site.

7. The conditionally activated binding protein of any one of claims 1-6, wherein the first binding domain comprises two or more polypeptides linked by a non-cleavable linker.

8. The conditionally activated binding protein of claim 6 or 7, wherein the binding protein is converted to the activated form upon a cleavage of the cleavable linker, and wherein in the activated form the second binding domain is separated from the first binding domain bound to the bulk serum protein, thereby removing the steric occlusion.

9. The conditionally activated binding protein of claim 8, wherein the binding protein is converted to the activated form in a protease rich environment.

10. The conditionally activated binding protein of any one of claims 1-9, wherein the first binding domain comprises a natural peptide, a synthetic peptide, an engineered scaffold, an engineered bulk serum protein, an immunoglobulin, any variants thereof, any fragments thereof, or a fusion protein comprising any combination thereof.

11. The conditionally activated binding protein of claim 10, wherein the engineered scaffold comprises at least one of: an sdAb, an scFv, an Fab, a VHH, a IgNAR, a VH, a VL, a fibronectin type III domain, an immunoglobulin-like scaffold, a bacterial albumin-binding domain, an adnectin, a monobody, an affibody, an affilin, an affimer, an affitin, an alphabody, an anticalin, an avimer, a centyrin, a DARPin, a cystine knot peptide, a lipocalin, a three-helix bundle scaffold, a protein G-related albumin-binding module, a DNA or RNA aptamer scaffold, or any combinations thereof.

12. The conditionally activated binding protein of any one of claims 1-11, wherein the first binding domain comprises a binding site specific for the bulk serum protein.

13. The conditionally activated binding protein of any one of claims 1-12, wherein the first binding domain comprises a binding site specific for an immunoglobulin light chain.

14. The conditionally activated binding protein of claim 13, wherein the immunoglobulin light chain is an Igκ free light chain.

15. The conditionally activated binding protein of any one of claims 12-14, wherein the first binding domain comprises one or more complementary determining regions (CDRs), and wherein the CDRs provide the binding site specific for the bulk serum protein or the immunoglobulin light chain.

16. The conditionally activated binding protein of any one of claims 1-15, wherein the first binding domain comprises a sequence selected from SEQ ID Nos.: 44-52.

17. The conditionally activated binding protein of any one of claims 1-16, wherein the second binding domain comprises an immunoglobulin molecule or a non-immunoglobulin molecule.

18. The conditionally activated binding protein of claim 17, wherein the second binding domain comprises an immunoglobulin molecule, wherein the immunoglobulin molecule is an antibody or an antibody fragment.

19. The conditionally activated binding protein of claim 18, wherein the second binding domain comprises a monoclonal antibody, a bispecific antibody, a chimeric antibody, a human antibody, a humanized antibody, a camelized antibody, or a variant thereof.

20. The conditionally activated binding protein of claim 19, wherein the second binding domain comprises the antibody fragment, and wherein the antibody fragment comprises a sdAb, Fab, Fab′-SH, Fv, scFv, (Fab′)2 fragment, a fragment of a chimeric antibody, a fragment of a bispecific antibody, or a variant thereof.

21. The conditionally activated binding protein of any one of claims 1-20, wherein in the inactive form the bulk serum protein is in close proximity to a binding site within the second binding domain, wherein the binding site is specific for the target.

22. The conditionally activated binding protein of any one of claims 1-21, wherein the target comprises a tumor antigen.

23. The conditionally activated binding protein of claim 22, wherein the tumor antigen comprises EpCAM, EGFR, HER-2, HER-3, c-Met, FoIR, PSMA, CD38, BCMA, and CEA. 5T4, AFP, B7-H3, Cadherin-6, CAIX, CD117, CD123, CD138, CD166, CD19, CD20, CD205, CD22, CD30, CD33, CD40, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, P-cadherin, NY-ESO-1, PRLR, PSCA, PTK7, ROR1, SLC44A4, SLTRK5, SLTRK6, STEAP1, TIM1, Trop2, or WT1.

24. The conditionally activated binding protein of any one of claims 1-21, wherein the target comprises an immune checkpoint protein.

25. The conditionally activated binding protein of claim 24, wherein the immune checkpoint protein comprises CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

26. The conditionally activated binding protein of any one of claims 1-21, wherein the target comprises an immune cell.

27. The conditionally activated binding protein of claim 26, wherein the immune cell comprises a T-cell.

28. The conditionally activated binding protein of any one of claims 1-21, wherein the target comprises CD3.

29. The conditionally activated binding protein of any one of claims 1-21, wherein the target comprises CD3ε.

30. The conditionally activated binding protein of any one of claims 1-29, wherein the first binding domain comprises two or more polypeptides linked by a non-cleavable linker.

31. The conditionally activated binding protein of any one of claims 6-30, wherein the protease cleavage site is recognized by a serine protease, a cysteine protease, an aspartate protease, a threonine protease, a glutamic acid protease, a metalloproteinase, a gelatinase, or a asparagine peptide lyase.

32. The conditionally activated binding protein of any one of claims 6-31, wherein the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP9, a MMP10, a MMP11, a MMP12, a MMP13, a MMP14, an ADAMS, an ADAM10, an ADAM12, an urokinase plasminogen activator (uPA), an enterokinase, a prostate-specific target (PSA, hK3), an interleukin-1β converting enzyme, a thrombin, a FAP (FAP-α), a dipeptidyl peptidase, a type II transmembrane serine protease (TTSP), a neutrophil elastase, a cathepsin G, a proteinase 3, a neutrophil serine protease 4, a mast cell chymase, and a mast cell tryptase.

33. A polynucleotide encoding the conditionally activated binding protein of any one of claims 1-32.

34. A vector comprising the polynucleotide of claim 33.

35. A host cell transformed with the vector according to claim 34.

36. A pharmaceutical composition comprising (i) the conditionally activated binding protein according to any one of claims 1-32, the polynucleotide according to claim 33, the vector according to claim 34, or the host cell according to claim 35 and (ii) a pharmaceutically acceptable carrier.

37. A process for the production conditionally activated binding protein of claim 36, said process comprising culturing a host transformed or transfected with a vector comprising a nucleic acid sequence.

38. A method for the treatment or amelioration of a proliferative disease or a tumorous disease, comprising the administration of conditionally activated binding protein of any one of claims 1-32 to a subject in need of such a treatment or amelioration.

39. The method according to claim 38, wherein the subject is a human.

40. The method according to claim 39, wherein the method further comprises administration of an agent in combination with the conditionally activated binding protein of any one of claims 1-32.


SEQUENCE LISTING (70 sequences)

SEQ ID NO: 1 - protein, 8 aa, Unknown
  Description of Unknown Protease cleavage site sequence
KRALGLPG

SEQ ID NO: 2 - protein, 40 aa, Unknown
  Description of Unknown Protease cleavage site sequence
DEDEDEDEDEDEDEDERPLALWRSDRDRDRDRDRDRDRDR

SEQ ID NO: 3 - protein, 5 aa, Unknown
  Description of Unknown Protease cleavage site sequence
  MOD_RES (3)..(3): Ser or Thr
  MOD_RES (4)..(4): Leu or Ile
  MOD_RES (5)..(5): Ser or Thr
PRXXX

SEQ ID NO: 4 - protein, 5 aa, Unknown
  Description of Unknown Protease cleavage site sequence
LEATA

SEQ ID NO: 5 - protein, 10 aa, Unknown
  Description of Unknown Protease cleavage site sequence
GGAANLVRGG

SEQ ID NO: 6 - protein, 10 aa, Unknown
  Description of Unknown Protease cleavage site sequence
SGRIGFLRTA

SEQ ID NO: 7 - protein, 6 aa, Unknown
  Description of Unknown Protease cleavage site sequence
PLGLAG

SEQ ID NO: 8 - protein, 6 aa, Unknown
  Description of Unknown Protease cleavage site sequence
  MOD_RES (6)..(6): Any amino acid
PLGLAX

SEQ ID NO: 9 - protein, 6 aa, Unknown
  Description of Unknown Protease cleavage site sequence
PLGCAG

SEQ ID NO: 10 - protein, 8 aa, Unknown
  Description of Unknown Protease cleavage site sequence
ESPAYYTA

SEQ ID NO: 11 - protein, 6 aa, Unknown
  Description of Unknown Protease cleavage site sequence
RLQLKL

SEQ ID NO: 12 - protein, 7 aa, Unknown
  Description of Unknown Protease cleavage site sequence
RLQLKAC

SEQ ID NO: 13 - protein, 7 aa, Unknown
  Description of Unknown Protease cleavage site sequence
  MOD_RES (3)..(3): Cit
  MOD_RES (5)..(5): Hof
EPXGXYL

SEQ ID NO: 14 - protein, 5 aa, Unknown
  Description of Unknown Protease cleavage site sequence
SGRSA

SEQ ID NO: 15 - protein, 4 aa, Unknown
  Description of Unknown Protease cleavage site sequence
DAFK

SEQ ID NO: 16 - protein, 5 aa, Unknown
  Description of Unknown Protease cleavage site sequence
GGGRR

SEQ ID NO: 17 - protein, 4 aa, Unknown
  Description of Unknown Protease cleavage site sequence
GFLG

SEQ ID NO: 18 - protein, 4 aa, Unknown
  Description of Unknown Protease cleavage site sequence
ALAL

SEQ ID NO: 19 - protein, 2 aa, Unknown
  Description of Unknown Protease cleavage site sequence
FK

SEQ ID NO: 20 - protein, 3 aa, Unknown
  Description of Unknown Protease cleavage site sequence
NLL

SEQ ID NO: 21 - protein, 5 aa, Unknown
  Description of Unknown Protease cleavage site sequence
PICFF

SEQ ID NO: 22 - protein, 8 aa, Unknown
  Description of Unknown Protease cleavage site sequence
GGPRGLPG

SEQ ID NO: 23 - protein, 6 aa, Unknown
  Description of Unknown Protease cleavage site sequence
HSSKLQ

SEQ ID NO: 24 - protein, 7 aa, Unknown
  Description of Unknown Protease cleavage site sequence
HSSKLQL

SEQ ID NO: 25 - protein, 9 aa, Unknown
  Description of Unknown Protease cleavage site sequence
HSSKLQEDA

SEQ ID NO: 26 - protein, 10 aa, Unknown
  Description of Unknown Protease cleavage site sequence
LVLASSSFGY

SEQ ID NO: 27 - protein, 10 aa, Unknown
  Description of Unknown Protease cleavage site sequence
GVSQNYPIVG

SEQ ID NO: 28 - protein, 10 aa, Unknown
  Description of Unknown Protease cleavage site sequence
GVVQASCRLA

SEQ ID NO: 29 - protein, 4 aa, Unknown
  Description of Unknown Protease cleavage site sequence
  MOD_RES (2)..(2): Pip
FXRS

SEQ ID NO: 30 - protein, 6 aa, Unknown
  Description of Unknown Protease cleavage site sequence
DPRSFL

SEQ ID NO: 31 - protein, 6 aa, Unknown
  Description of Unknown Protease cleavage site sequence
PPRSFL

SEQ ID NO: 32 - protein, 4 aa, Unknown
  Description of Unknown Protease cleavage site sequence
DEVD

SEQ ID NO: 33 - protein, 5 aa, Unknown
  Description of Unknown Protease cleavage site sequence
DEVDP

SEQ ID NO: 34 - protein, 8 aa, Unknown
  Description of Unknown Protease cleavage site sequence
KGSGDVEG

SEQ ID NO: 35 - protein, 6 aa, Unknown
  Description of Unknown Protease cleavage site sequence
GWEHDG

SEQ ID NO: 36 - protein, 7 aa, Unknown
  Description of Unknown Protease cleavage site sequence
EDDDDKA

SEQ ID NO: 37 - protein, 9 aa, Unknown
  Description of Unknown Protease cleavage site sequence
KQEQNPGST

SEQ ID NO: 38 - protein, 6 aa, Unknown
  Description of Unknown Protease cleavage site sequence
GKAFRR

SEQ ID NO: 39 - protein, 4 aa, Unknown
  Description of Unknown Protease cleavage site sequence
DAFK

SEQ ID NO: 40 - protein, 4 aa, Unknown
  Description of Unknown Protease cleavage site sequence
DVLK

SEQ ID NO: 41 - protein, 4 aa, Unknown
  Description of Unknown Protease cleavage site sequence
DAFK

SEQ ID NO: 42 - protein, 7 aa, Unknown
  Description of Unknown Protease cleavage site sequence
ALLLALL

SEQ ID NO: 43 - protein, 115 aa, Homo sapiens [sequence omitted]

SEQ ID NO: 44 - protein, 115 aa, Artificial Sequence
  Description of Artificial Sequence Synthetic polypeptide
EVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 45 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGADTLYADSLKGRFTISRDNAKTTLYLQMNSLRPEDTAVYYCTIGGSLSKSSQGTLVTVSS

SEQ ID NO: 46 - protein, 115 aa, Artificial Sequence
  Description of Artificial Sequence Synthetic polypeptide
EVQLVESGGGLVQPGNSLRLSCAASGFTYSSFGMSWVRQAPGKGLEWVSSISGSGSDTLYADSVKGRFTISRDNAKTTLYLQMNSLRPEDTAVYYCTIGGSLSKSSQGTLVTVSS

SEQ ID NO: 47 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGTDTLYADSVKGRFTISRDNAKTTLYLQMNSLRPEDTAVYYCTIGGSLSRSSQGTLVTVSS

SEQ ID NO: 48 - protein, 115 aa, Artificial Sequence
  Description of Artificial Sequence Synthetic polypeptide
EVQLVESGGGLVQPGNSLRLSCAASGFTFSRFGMSWVRQAPGKGLEWVSSISGSGSDTLYADSVKGRFTISRDNAKTTLYLQMNSLRPEDTAVYYCTIGGSLSKSSQGTLVTVSS

SEQ ID NO: 49 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 50 - protein, 115 aa, Artificial Sequence
  Description of Artificial Sequence Synthetic polypeptide
EVQLVESGGGLVQPGNSLRLSCAASGFTFSRFGMSWVRQAPGKGLEWVSSISGSGSDTLYAESVKGRFTISRDNAKTTLYLQMNSLRPEDTAVYYCTIGGSLSRSSQGTLVTVSS

SEQ ID NO: 51 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGTDTLYAESVKGRFTISRDNAKTTLYLQMNSLRPEDTAVYYCTIGGSLSRSSQGTLVTVSS

SEQ ID NO: 52 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 53 - protein, 115 aa, Artificial Sequence
  Description of Artificial Sequence Synthetic polypeptide
EVQLVESGGGLVQPGNSLTLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 54 - protein, 19 aa, Artificial Sequence
  Description of Artificial Sequence Synthetic peptide
GGGGKPLGLQARVVGGGGT

SEQ ID NO: 55 - protein, 249 aa, Artificial Sequence
  Description of Artificial Sequence Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 56 - protein, 9 aa, Artificial Sequence
  Description of Artificial Sequence Synthetic peptide
GGGGSGGGS

SEQ ID NO: 57 - protein, 133 aa, Artificial Sequence
  Description of Artificial Sequence Synthetic polypeptide
EVQLVESGGGLVQPGGSLRLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 58 - protein, 249 aa, Artificial Sequence
  Description of Artificial Sequence Synthetic polypeptide
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 59 - protein, 372 aa, Artificial Sequence
  Description of Artificial Sequence Synthetic polypeptide
MALPVTALLLPLALLLHAARPEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 504 aa, Artificial Sequence
  Description of Artificial Sequence Synthetic polypeptide
MALPVTALLLPLALLLHAARPEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 368 aa, Artificial Sequence
  Description of Artificial Sequence Synthetic polypeptide
MALPVTALLLPLALLLHAARPQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 10 aa, Unknown
  Description of Unknown Protease cleavage site sequence
KPLGLQARVV

SEQ ID NO: 63 - protein, 10 aa, Unknown
  Description of Unknown Protease cleavage site sequence
PQASTGRSGG

SEQ ID NO: 64 - protein, 10 aa, Unknown
  Description of Unknown Protease cleavage site sequence
PQGSTGRAAG

SEQ ID NO: 65 - protein, 10 aa, Unknown
  Description of Unknown Protease cleavage site sequence
PPASSGRAGG

SEQ ID NO: 66 - protein, 9 aa, Unknown
  Description of Unknown Protease cleavage site sequence
PIPVQGRAH

SEQ ID NO: 67 - protein, 10 aa, Unknown
  Description of Unknown Protease cleavage site sequence
PQGSTARSAG

SEQ ID NO: 68 - protein, 8 aa, Unknown
  Description of Unknown metalloproteinase cleavage site
GPLGIAGQ

SEQ ID NO: 69 - protein, 8 aa, Unknown
  Description of Unknown metalloproteinase cleavage site
AVRWLLTA

SEQ ID NO: 70 - protein, 6 aa, Unknown
  Description of Unknown furin cleavage site
RRRRRR